(19) United States
(12) Patent Application Publication
(10) Pub. No.: US 2018/0045698 A1
(43) Pub. Date: Feb. 15, 2018

(54) ENHANCING SENSITIVITY BY DIRECTLY PRINTING NANOSENSORS USING ADVANCED MANUFACTURING TECHNIQUES ON A PRE-AMP BOARD OR A DAUGHTERBOARD

(71) Applicant: U.S.A. as represented by the Administrator of the National Aeronautics and Space Administration, Washington, DC (US)

(72) Inventor: MAHMOODA SULTANA, Laurel, MD (US)

(21) Appl. No.: 15/234,222

(22) Filed: Aug. 11, 2016

(51) Int. Cl. G01N 33/00; G01N 31/22; G01N 27/04

(52) U.S. Cl. CPC G01N 33/0027; G01N 27/04; G01N 31/223

(57) ABSTRACT

The present invention provides a printable nanosensor and leads that can be manufactured using three-dimensional printing techniques and printing directly on a daughter board that can be connected to a self-contained pre-amplified printed circuit board. Although graphene is highly sensitive as a chemical sensor and can detect parts per million (ppm) concentrations of species, the integration of printed graphene and other two-dimensional sensing materials to form a sensor assembly can be used to increase the sensitivity even further, enabling detection of parts per billion (ppb) concentrations and even single molecule detection.

### STATEMENT REGARDING FEDERALLY SPONSORED RESEARCH OR DEVELOPMENT

The invention described herein was made in part by employees of the United States Government and may be manufactured and used by or for the Government of the United States of America for governmental purposes without the payment of any royalties thereon or therefor.

### FIELD OF THE INVENTION

The present invention relates to enhancing sensitivity of chemical sensors by printing or fabricating the sensors using additive manufacturing techniques directly on a self-contained pre-amplified printed circuit board or on a daughterboard that can be integrated to a self-contained pre-amplified printed circuit board.

### BACKGROUND OF THE INVENTION

Contamination and outgassing can often degrade the performance of optics in space. Due to the lack of low cost methods, the sources of contamination may remain undiagnosed. An example of such a problem is where the performance of optics of a spacecraft is degrading over time and the sources of the degradation are unknown, leaving no scope to correct the issues in future missions. There is a need for small, inexpensive, and sensitive sensors that can address this issue and contribute toward the success of future missions. Furthermore, there is a need for small, light, and sensitive chemical sensors that can be used for future human exploration missions, where the ability to detect toxic gases, such as hydrazine in spacecrafts, will be necessary to ensure human safety and mission success.

### SUMMARY OF THE INVENTION

According to the present invention, a chemical sensor assembly is provided that comprises one or more one-dimensional or two-dimensional material, chemical sensors that are three-dimensionally printed, nanoimprinted, or transferred onto a printed circuit board (PCB), for example, directly onto the PCB or on a daughterboard. The two-dimensional materials can comprise one, two, or more different chemical-sensing materials that are formed on separate respective areas of the printed circuit board. Gas molecules present in an atmosphere exposed to the chemical sensor assembly can react or adsorb onto each of the different chemical-sensing sensors. In an exemplary embodiment, an array of graphene sensors is printed on a printed circuit board, an array of carbon nanotube (CNT) sensors are printed on the same printed circuit board, adjacent to the array of graphene sensors, and an array of molybdenum disulfide sensors are printed on the same printed circuit board, adjacent to the arrays of graphene and carbon nanotube sensors.

One of the most useful applications of two-dimensional materials such as graphene is its ability to act as a chemical sensor. Due to the fact that practically the entire volume of the two-dimensional structure is exposed to the surrounding atmosphere, such two-dimensional structures and materials provide an efficient and highly sensitive chemical sensor. In addition, the material has exceptional electrical, chemical, thermal, and mechanical properties. Graphene is also a radiation hard material due to its minute cross-section, which makes it ideal for space applications. The electrical properties of graphene often change upon either adsorbing or reacting with molecules. Thus, graphene can be used as a gas sensor for many disciplines, including planetary sciences, earth sciences, heliophysics and terrestrial applications. Graphene chemical sensors make extremely sensitive detectors for in-situ measurement of atomic species such as atomic oxygen. An integrated nanosensor according to the present invention can be printed on a daughter board using three-dimensional printing techniques, and the daughter board can already be or subsequently be directly wire-bonded to a self-contained pre-amplifying printed circuit board.

The chemical sensor assemblies of the present invention can use field effect transistors, simple resistors or other device structures based on two-dimensional materials to sense the surface potential of a graphene channel exposed to an analyte. In one embodiment, when analyte molecules adsorb onto the sensor surface, for example, onto the surface of graphene, they act as electron donors or acceptors, inducing a local change in electrical resistance. This effect is very pronounced in two-dimensional materials due to high surface area, high electrical conductivity (at least in the case of graphene), and inherent low noise, making it possible to detect changes in resistance. Different gases may have different effects on the resistivity. The magnitude and the sign of the change indicate whether the adsorbed gas molecule is an acceptor, for example, NO₂ or H₂O, or a donor, for example, CO or NH₃. The selectivity among target gases can be improved further through functionalization of the nano materials. Functional groups attached to graphene, for example, can be chosen that selectively interact with target molecules. The two-dimensional chemical sensor material can be microfabricated on a suitable substrate, for example, as arrays, to form a sensor assembly. By using different 1-dimensional and two-dimensional chemical sensors, and/or functional groups with them in an array, the sensor assembly can provide a suite of sensors with different chemical interactions for targeting different analytes.

The chemical sensor assembly can contain a silicon daughter board having a single chemical sensor, or one or more arrays of chemical sensors, printed, transferred, or otherwise formed thereon. The chemical sensors can comprise, for example, a printed graphene sensor array, a printed carbon nanotube sensor array, a printed molybdenum disulfide sensor array, or a combination thereof, printed directly on a printed circuit board or daughter board having a pre-amplifier circuit connected thereto. The circuit can be connected to the board with pins, wire bonds, or a combination thereof. If the chemical sensors are printed, transferred, or otherwise formed on a daughter board, the daughter board can thereafter or firstly be plugged into or otherwise connected to a mother board. The sensor dimensions can be of any size, for example, from one or a few microns to 100s of microns or more.

### BRIEF DESCRIPTION OF THE DRAWINGS

The present invention can be even more fully understood with the reference to the accompanying drawings which are intended to illustrate, not limit, the present invention.

FIG. 1 is a schematic diagram showing a two-dimensional graphene chemical sensor connected to a gold, current-in (Iᵢₙ) electrical lead at one end thereof and connected to a gold, current-out (Iₒᵤₜ) electrical lead at the other end thereof, and showing a second pair of gold leads used to measure a change in resistivity across the two-dimensional graphene material, resulting from the adsorption of or interaction with a gas molecule.

FIG. 2 shows a chemical sensor assembly according to various embodiments of the present invention, comprising an array of carbon nanotube (CNT) chemical sensors, an array of molybdenum disulfide (MoS₂) chemical sensors, and an array of graphene chemical sensors, which arrays have been 3D printed directly on the insulating materials of the printed circuit board.

FIG. 3 is a schematic diagram of a molecule (molecule 1) and a bar graph showing the change in electrical resistivity resulting from the adsorption of molecule 1 on a chemical sensor of the CNT array (Array A), the adsorption of molecule 1 on a chemical sensor of the MoS₂ array (Array B), and the adsorption of molecule 1 on a chemical sensor of the graphene array (Array C), whereby the chemical structure of molecule 1 can be discerned from the resulting spectrum or fingerprint obtained from a curve drawn on the bar graph.

FIG. 4 is a schematic diagram of a molecule (molecule 2) and a bar graph showing the change in electrical resistivity resulting from the adsorption of molecule 2 on a chemical sensor of the CNT array (Array A), the adsorption of molecule 2 on a chemical sensor of the MoS₂ array (Array B), and the adsorption of molecule 2 on a chemical sensor of the graphene array (Array C), whereby the chemical structure of molecule 2 can be discerned from the resulting spectrum or fingerprint obtained from a curve drawn on the bar graph.

### DETAILED DESCRIPTION OF THE INVENTION

According to the present invention a chemical sensor assembly is provided that comprises a printed circuit board (PCB) and a two-dimensional ultrathin film chemical sensor structure. The PCB comprises at least a first pair of electrical leads and can have one or pairs of electrical leads or one or more arrays of pairs of electrical leads. The two-dimensional ultrathin film chemical sensor structure can be printed onto the printed circuit board using additive manufacturing or 3-D printing techniques, using a first chemical sensor material, for example, graphene. The first pair of electrical leads are electrically connected to the two-dimensional ultrathin film chemical sensor structure. A current can be run through the structure and a second pair of electrical leads can be used to sense increases and decreases in electrical resistivity across the structure.

The chemical sensor assembly can comprise a first array of chemical sensors. The two-dimensional ultrathin film chemical sensor structure can comprise a first array of chemical sensors. Each chemical sensor of the first array can comprise a two-dimensional ultrathin film chemical sensor structure printed onto the printed circuit board. Each chemical sensor of the first array can be configured, for example, wired, to be in electrical contact with respective electrical leads of the printed circuit board.

The chemical sensor assembly can comprise a first and a second two-dimensional ultrathin film chemical sensor structures printed onto the printed circuit board using additive manufacturing techniques. The printed circuit board can comprise first and second pairs of electrical leads. The second two-dimensional ultrathin film chemical sensor structure can comprise a different material than the first chemical sensor material. The second two-dimensional ultrathin film chemical sensor structure can be electrically connected to the second pair of electrical leads.

The chemical sensor assembly can comprise first and second arrays of chemical sensors. Each array can be printed onto the printed circuit board using additive manufacturing techniques such as three dimensional printing, aerojet printing, or stamping. The second array can be made of or include a second chemical sensor material that differs from the first chemical sensor material. The printed circuit board can further comprise a first array of electrical lead pairs that are electrically connected, respectively, to the first array of chemical sensors, and a second array of electrical lead pairs, that are electrically connected, respectively, to the second array of chemical sensors.

The chemical sensor assembly can further comprise a third array of chemical sensors printed onto the printed circuit board using additive manufacturing techniques. The third array can comprise a third chemical sensor material that differs from the first chemical sensor material and differs from the second chemical sensor material. The printed circuit board can comprise a third array of electrical lead pairs that are electrically connected, respectively, to the third array of chemical sensors.

The two-dimensional ultrathin film chemical sensor structure can comprise any suitable material, for example, graphene, a carbon nanotube material, molybdenum disulfide, a combination thereof, or the like. For example, the first array of chemical sensors can comprise graphene. The first array of chemical sensors can comprise graphene while the second array of chemical sensors can comprise a carbon nanotube material and the third array of chemical sensors can comprise molybdenum disulfide. The first chemical sensor material comprises graphene and the two-dimensional ultrathin film chemical sensor structure comprises a single molecule thickness of graphene.

The printed circuit board can be electrically connected to a pre-amplification electrical circuit. The chemical sensing structures can be printed onto a mother board having integrated therein a pre-amplification circuit, or can be printed onto a daughter board for subsequent integration with a mother board having a pre-amplification circuit.

The present invention also provides a method of making a chemical sensor assembly. The method comprises three-dimensionally printing a two-dimensional ultrathin film chemical sensor structure onto a surface of a printed circuit board. The two-dimensional chemical sensor structure can be electrically connected to a pair of electrical leads during or after printing. The leads can be configured for applying current to the two-dimensional ultrathin film chemical sensor structure. The method can comprise electrically connecting the two-dimensional ultrathin film chemical sensor structure to a second pair of electrical leads configured for measuring electrical resistance across the two-dimensional ultrathin film chemical sensor structure. The method can be used to form a daughter board, and the method can further comprise connecting the daughter board to a mother board that comprises a pre-amplification electrical circuit.

The method can comprise three-dimensionally printing a first material to form a first array of chemical sensors onto the printed circuit board. The first array of chemical sensors can comprise an array of two-dimensional ultrathin film chemical sensor structures including the two-dimensional ultrathin film chemical sensor structure described above. The method can further include three-dimensionally printing a second array of chemical sensors onto the printed circuit board. The second array of chemical sensors can comprise an array of second two-dimensional ultrathin film chemical sensor structures, each of which comprises a material that differs from the material of the first array of second two-dimensional ultrathin film chemical sensor structures. The method can further comprise three-dimensionally printing a third array of chemical sensors onto the printed circuit board. The third array of chemical sensors can comprise an array of third two-dimensional ultrathin film chemical sensing structures, each of which comprises a third material that differs from the material of the first array and that differs from the material of the second array.

The present invention also provides a method of sensing the presence of a gas. The method comprises exposing a gas to a chemical sensor assembly of the present invention and adsorbing the gas onto each of the first array of chemical sensors, the second array of chemical sensors, and the third array of chemical sensors. Herein, adsorbing can include an interaction whereby with such that a change in electrical properties of the chemical sensor structure results. Any such change can be measured according to the method, for example, a change in electrical resistivity across each two-dimensional ultrathin film chemical sensor structure of the first array, the second array, and the third array. The change can be attributable to adsorption of the gas on one or more of the structures of the respective array. Thereafter, a resistance spectrum can be generated from the change in electrical resistivity measured, and the resistance spectrum can be compared to known resistance spectra to determine the chemical make-up of the gas. Known resistance spectra can be stored in a datastore, downloaded, printed out for visual inspection, or the like. The method can be used with an assembly as described herein, for example, and assembly wherein the first array of chemical sensors comprises graphene, the second array of chemical sensors comprises a carbon nanotube material, and the third array of chemical sensors comprises molybdenum disulfide.

According to various embodiments of the present invention, sensor assemblies comprising highly sensitive, two-dimensional sensors are suitable for a wide range of uses, including mission architectures in Planetary Sciences, atmospheric probes, and landed missions. The versatility of the present sensor assemblies with respect to operating conditions and their ability to be customized and to detect selected gases of interest makes such assemblies useful for a broad range of missions. The sensor assemblies can detect gases that can help fingerprint various biological and geochemical processes on other planetary bodies such as outer planets, moons, and asteroids. More specifically, the sensors can be used to detect methane, carbon monoxide, and other gases that are difficult to detect using mass spectrometry because of mass interference, including interference by volatile organic compounds such as formaldehyde and formic acid that are present on Mars. In Heliophysics, small and fast graphene or molydisulfide (molybdenum disulfide) sensors offer enhanced time resolution of thermosphere physics investigations in low-Earth-orbit missions. Graphene sensors can be used to identify neutral molecules and measure density in the thermosphere directly, without having to ionize such neutral molecules. The ability to determine neutral densities can provide an avenue to significantly resolve the density measurement uncertainty, which in turn can improve the density models used by NASA and DOD.

Designed as an atomic oxygen detector, the chemical sensor assembly of the present invention can be used in series with Stark effect to make a powerful tool to separate and detect species. Additionally, the chemical sensor assembly can be used in neutral atom imaging missions such as the Low Energy Neutral Atom (LENA) mission. The information on the atomic oxygen densities can also be used to predict geomagnetically induced storms. Moreover, small and inexpensive two-dimensional chemical sensor assemblies can be used in contamination control and monitoring, as well as in the remote sensing of trace gases.

Due to the two-dimensionality of the materials of the chemical sensors, the chemical sensors have the highest possible surface-to-volume ratio because all of the atoms of the two-dimensional structure are exposed at the surface. This enables great sensitivity. Moreover, the chemical sensors, chemical sensor arrays, and sensor assemblies of the present invention exhibit: radiation hardness due to a minute cross sections; a high chemical, mechanical, and thermal stability; a low Johnsons noise; a high mobility, particularly in the case of graphene; and the capability of performing in-situ detection of trace gases and atomic oxygen.

By forming the sensors on a daughter board and providing pre-amplification electronics, sensor signals can be significantly increased, and thus the sensitivity to and detection of minute concentrations of gases are enabled. Chemical concentrations as low as a few parts per billion, and even single molecule detection, is enabled. In addition, the self-contained printed circuit board eliminates a number of interfaces and thus eliminates a number of sources of error, resulting in even further increased sensitivity. The two-dimensional chemical sensors and sensor assemblies including them, of the present invention, have applications in trace gas sensing and so are useful in both planetary sciences and earth sciences. The sensor assemblies also have applications in contamination control and heliophysics.

Exemplary ultrathin film materials that can be printed and used according to the present invention include not only graphene, molybdenum disulfide, and carbon nanotubes, but can also or instead include MgO/Mo, MgO/Mo(100), MgO/Ag(100), NiO/Ni(100), Fe/MgO/Fe, SiO₂/Ru(0001) two-layer materials, SiO₂/graphene, FeO/Pt(111), Y₂O₃/ZrO₂, CaF₂/BaF₂, and ultrathin films made of La₂GeO₅, SiO₂, ZnO, WO₃, SnO₂, LaTiO₃, and Bi₂Sr₂Ca₂Cu₃₀O₁₀. Other materials that can be printed and used to form ultrathin films according to the present invention include those described, for example, in Pachioni, “Two-Dimensional Oxides: Multifunctional Materials for Advanced Technologies,” from Chem. Eur. J. 2012, 18, 10144-10158, Wiley-VCH Verlag GmbH & Co. KGaA, Weinheim, which is incorporated herein in its entirety by reference. Herein, by ultrathin films, what is meant is two-dimensional materials, for example, two-dimensional oxides, that are formed to a thickness of 50 nanometers or less, and that are grown on another substrate such as a metal, a semiconductor, or an insulator. Other two-dimensional materials that can be used to form chemical sensors and chemical sensor arrays according to the present invention include the metal dichalcogenides described by Sorkin in “Nanoscale Transition Metal Dichalcogenides: Structures, Properties, and Applications,” Journal: Critical reviews in solid state and materials sciences, ISSN: 1040-8436, September 2014, Volume: 39, Issue: 5, Pages 319-367, DOI: 10.1080/10408436.2013.863176, which is incorporated herein in its entirety by reference.

While any suitable printing technique can be used to form the sensors on a daughter board or printed circuit board, one particularly useful technique for forming the two-dimensional sensors comprises the use of a three-dimensional printer. Ink jet, laser jet, bubble jet, ink nozzle, and other printing techniques can alternatively or additionally be used. Element transfer technologies can be used to manufacture the sensors, for example, using nanoelement transfer methods, damascene templates, or a combination thereof as described, for example, in U.S. Pat. No. 9,365,946 B2 to Busnaina et al., which is incorporated herein in its entirety by reference. The sensors can be manufactured by electric field driven nanoelement assembly techniques such as those described, for example, in U.S. Pat. No. 9,145,618 B2 to Sirman et al., and in U.S. Patent Application Publication No. US 2016/0021738 A1 to Sirman et al., both of which are incorporated herein in their entireties by reference.

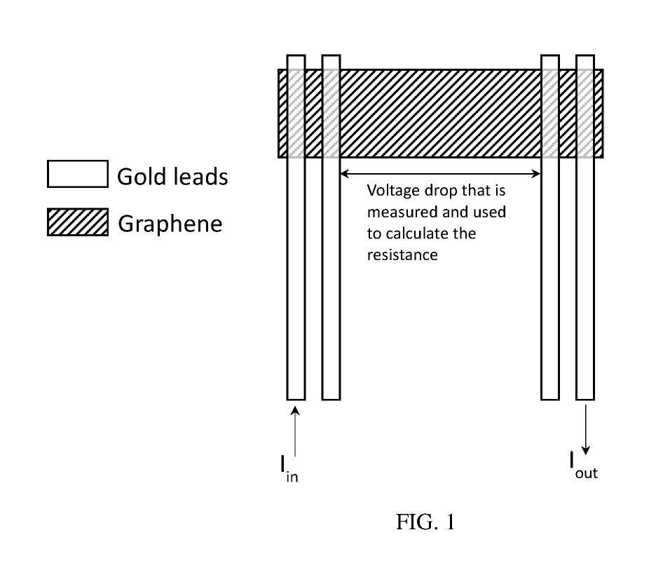

With reference to the drawings, FIG. 1 is a schematic diagram showing a two-dimensional graphene chemical sensor connected to a gold, current-in (Iᵢₙ) electrical lead at one end thereof and connected to a gold, current-out (Iₒᵤₜ) electrical lead at the other end thereof. FIG. 1 also shows a second pair of gold leads that are used to measure a change in electrical resistivity across the two-dimensional graphene material. Changes in resistivity across the material can be detected and result from the adsorption of a gas molecule on the material or an interaction between the material and a gas molecule.

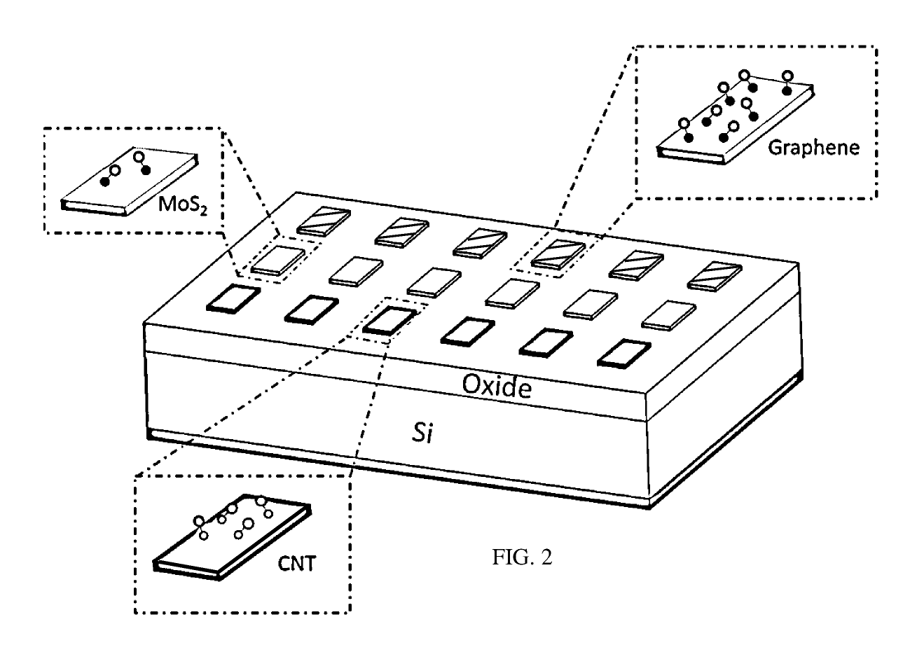

FIG. 2 shows a chemical sensor assembly according to various embodiments of the present invention, comprising an array of carbon nanotube (CNT) chemical sensors, an array of molybdenum disulfide (MoS₂) chemical sensors, and an array of graphene chemical sensors. The arrays have an been three-dimensionally printed directly on the oxide surface of a printed circuit board.

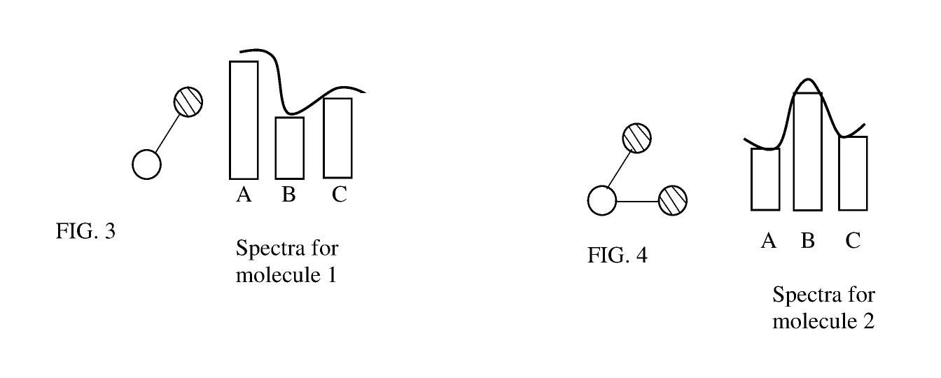

FIG. 3 is a schematic diagram of a molecule (molecule 1) and a bar graph showing the change in electrical resistivity resulting from the adsorption of molecule 1 on a chemical sensor of the CNT array (Array A), the adsorption of molecule 1 on a chemical sensor of the MoS₂ array (Array B), and the adsorption of molecule 1 on a chemical sensor of the graphene array (Array C). Due to the very specific change of resistivity resulting from the adsorption of molecule 1 on each of the different types of sensors, the chemical structure of molecule 1 can be discerned. The resulting spectrum or fingerprint obtained from the curve drawn on the bar graph, can be compared to known spectra to determine the chemical make-up of molecule 1.

FIG. 4 is a schematic diagram of a molecule (molecule 2) and a bar graph showing the change in electrical resistivity resulting from the adsorption of molecule 2 on a chemical sensor of the CNT array (Array A), the adsorption of molecule 2 on a chemical sensor of the MoS₂ array (Array B), and the adsorption of molecule 2 on a chemical sensor of the graphene array (Array C). The chemical structure of molecule 2 can be discerned from the resulting spectrum or fingerprint obtained from the curve drawn on the bar graph and a comparison of that spectrum to other, known spectra.

In accordance with the present invention, the small, light, and sensitive sensors of the present invention can be used for chemical analysis on remote terrestrial areas, as well as other planetary bodies such as planets, moons, asteroids, and comets. They can be used to prescreen samples for sample return missions, as well as to monitor outgassing of collected samples during storage in sample return missions. They can be used to detect toxic gases, such as hydrazine, in spacecrafts. They can be used to monitor process quality in the chemical industry, for hazardous and/or toxic gas detection in chemical plants, for monitoring environmental pollutants, for detecting explosives at public places such as airports, schools, and courthouses. They can be used for medical diagnoses. The graphene chemical sensors of the present invention can be used as extremely sensitive detectors for in-situ measurement of atomic species such as atomic oxygen.

The entire contents of all references cited in this disclosure are incorporated herein in their entireties, by reference. Herein, the term “about” means within a range of from plus 5% to minus 5% the value modified. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether such ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

Other embodiments of the present invention will be apparent to those skilled in the art from consideration of the present specification and practice of the present invention disclosed herein. It is intended that the present specification and examples be considered as exemplary only with a true scope and spirit of the invention being indicated by the following claims and equivalents thereof.

What is claimed is:

1. A chemical sensor assembly comprising:
a printed circuit board comprising a first pair of electrical leads; and
a two-dimensional ultrathin film chemical sensor structure three-dimensionally printed onto the printed circuit board from a first chemical sensor material, wherein the first pair of electrical leads are electrically connected to the two-dimensional ultrathin film chemical sensor structure.

2. The chemical sensor assembly of claim 1, further comprising a first array of chemical sensors, wherein the two-dimensional ultrathin film chemical sensor structure comprises a first chemical sensor of the first array of chemical sensors, each chemical sensor of the first array comprises a two-dimensional ultrathin film chemical sensor structure printed onto the printed circuit board, and each chemical sensor of the first array is in electrical contact with respective electrical leads of the printed circuit board.

3. The chemical sensor assembly of claim 1, further comprising a second two-dimensional ultrathin film chemical sensor structure printed onto the printed circuit board using additive manufacturing techniques, wherein the printed circuit board comprises a second pair of electrical leads, the second two-dimensional ultrathin film chemical sensor structure comprises a different material than the first chemical sensor material, and the second two-dimensional ultrathin film chemical sensor structure is electrically connected to the second pair of electrical leads.

4. The chemical sensor assembly of claim 2, further comprising a second array of chemical sensors printed onto the printed circuit board using additive manufacturing techniques and that comprises a second chemical sensor material that differs from the first chemical sensor material, wherein the printed circuit board further comprises a first array of electrical lead pairs that are electrically connected, respectively, to the first array of chemical sensors, and a second array of electrical lead pairs, that are electrically connected, respectively, to the second array of chemical sensors.

5. The chemical sensor assembly of claim 4, further comprising a third array of chemical sensors three-dimensionally printed onto the printed circuit board and that comprises a third chemical sensor material that differs from the first chemical sensor material and differs from the second chemical sensor material, wherein the printed circuit board further comprises a third array of electrical lead pairs that are electrically connected, respectively, to the third array of chemical sensors.

6. The chemical sensor assembly of claim 1, wherein the two-dimensional ultrathin film chemical sensor structure comprises graphene, a carbon nanotube material, molybdenum disulfide, or a combination thereof.

7. The chemical sensor assembly of claim 2, wherein the first array of chemical sensors comprises a single layer, bilayer, or multilayer graphene.

8. The chemical sensor assembly of claim 5, wherein the first array of chemical sensors comprises graphene, the second array of chemical sensors comprises a carbon nanotube material, and the third array of chemical sensors comprises molybdenum disulfide.

9. The chemical sensor assembly of claim 1, wherein the printed circuit board is electrically connected to a pre-amplification electrical circuit.

10. The chemical sensor assembly of claim 1, wherein the first chemical sensor material comprises graphene and the two-dimensional ultrathin film chemical sensor structure comprises a single molecule thickness of graphene.

11. A method of making a chemical sensor assembly, comprising:
three-dimensionally printing a two-dimensional ultrathin film chemical sensor structure onto a surface of a printed circuit board;
electrically connecting the two-dimensional chemical sensor structure to a pair of electrical leads configured for applying current to the two-dimensional ultrathin film chemical sensor structure; and
electrically connecting the two-dimensional ultrathin film chemical sensor structure to a second pair of electrical leads configured for measuring electrical resistance across the two-dimensional ultrathin film chemical sensor structure.

12. The method of claim 11, wherein the printed circuit board comprises a daughter board, and the method further comprises connecting the daughter board to a mother board that comprises a pre-amplification electrical circuit.

13. The method of claim 11, further comprising three-dimensionally printing a first material to form a first array of chemical sensors onto the printed circuit board, the first array of chemical sensors comprising an array of two-dimensional ultrathin film chemical sensor structures including the two-dimensional ultrathin film chemical sensor structure.

14. The method of claim 13, further comprising three-dimensionally printing a second array of chemical sensors onto the printed circuit board, the second array of chemical sensors comprising an array of second two-dimensional ultrathin film chemical sensor structures, each of which comprises a material that differs from the first material.

15. The method of claim 14, further comprising three-dimensionally printing a third array of chemical sensors onto the printed circuit board, the third array of chemical sensors comprising an array of third two-dimensional ultrathin film chemical sensing structures, each of which comprises a third material that differs from the first material and that differs from the second material.

16. A method of sensing the presence of a gas, comprising
exposing a gas to a chemical sensor assembly made by the method of claim 15;
adsorbing the gas onto each of the first array of chemical sensors, the second array of chemical sensors, and the third array of chemical sensors;
measuring the change in electrical properties such as resistivity attributable to adsorption of the gas, across each two-dimensional ultrathin film chemical sensor structure of the first array, the second array, and the third array;
generating a resistance spectrum from the change in electrical resistivity measured; and
comparing the resistance spectrum to known resistance spectra to determine the chemical make-up of the gas.

17. The method of claim 16, wherein the first array of chemical sensors comprises graphene, the second array of chemical sensors comprises a carbon nanotube material, and the third array of chemical sensors comprises molybdenum disulfide.
